(19) United States
(12) Patent Application Publication
(10) Pub. No.: US 2013/0101580 A1
(43) Pub. Date: Apr. 25, 2013

(54) COMPOSITIONS AND METHODS FOR PROLONGING LIFESPAN

(71) Applicants: Gregory Oxenkrug, Newton, MA (US); Paul Summergrad, Newton Centre, MA (US)

(72) Inventors: Gregory Oxenkrug, Newton, MA (US); Paul Summergrad, Newton Centre, MA (US)

(21) Appl. No.: 13/659,427

(22) Filed: Oct. 24, 2012

Related U.S. Application Data
(60) Provisional application No. 61/550,652, filed on Oct. 24, 2011.

(51) Int. Cl. A61K 31/405; A61K 39/395; A61P 9/00; A61P 25/00; A61P 21/00; A61P 3/04; A61P 19/08; A61P 25/20; A61P 15/10; A61P 17/00; C12Q 1/26; C12Q 1/02; G01N 33/566; C12Q 1/68; C40B 30/06; A61K 31/7088

(52) U.S. Cl. 424/130.1; 514/419; 514/44 R; 435/25; 435/29; 435/7.21; 435/6.13; 506/10

(57) ABSTRACT

The present invention relates to therapeutic targets for aging. In particular, the present invention relates to the inhibition of the kynurenine pathway of tryptophan metabolism to extend lifespan or provide anti-aging benefits.

This application claims priority to U.S. Provisional Patent Application Ser. No. 61/550,652, filed on Oct. 24, 2011, which is herein incorporated by reference in its entirety

### FIELD OF THE INVENTION

The present invention relates to therapeutic targets for aging. In particular, the present invention relates to the inhibition of the kynurenine pathway of tryptophan metabolism to extend lifespan or provide anti-aging benefits.

### BACKGROUND OF THE INVENTION

Aging-associated medical and psychiatric disorders affect multiple systems. Typical disorders of aging include reduced cardiac output, mood & cognitive changes, muscle wasting, decreased energy, abdominal fat/truncal obesity, weak bones, thin skin and skin wrinkles, poor sleep and lessened sexual performance.

For example, over time, the heart muscle becomes less efficient, working harder to pump the same amount of blood. In addition, blood vessels lose some of their elasticity and hardened fatty deposits may form on the inner walls of arteries (atherosclerosis). These changes make arteries stiffer, causing the heart to work even harder to pump blood through them. This can lead to high blood pressure (hypertension) and other cardiovascular problems.

With age, bones tend to shrink in size and density, which weakens them and makes them more susceptible to fracture. Muscles generally lose strength and flexibility, and individuals may become less coordinated or have trouble balancing.

In addition, constipation is more common in older adults. Many factors can contribute to constipation, including a low-fiber diet, not drinking enough fluids and lack of exercise. Various medications, including diuretics and iron supplements, may contribute to constipation. Certain medical conditions, including diabetes and irritable bowel syndrome, may increase the risk of constipation as well.

Loss of bladder control (urinary incontinence) is common with aging. Health problems such as obesity, frequent constipation and chronic cough may contribute to incontinence, as can menopause, for women, and an enlarged prostate, for men.

Further, memory tends to becomes less efficient with age, as the number of cells (neurons) in the brain decreases. It may take longer to learn new things or remember familiar words or names.

With age, the eyes are less able to produce tears, the retinas thin, and the lenses gradually become less clear. Focusing on objects that are close up may become more difficult. People may become more sensitive to glare and have trouble adapting to different levels of light. Hearing may dim somewhat as well, in particular hearing high frequencies or following a conversation in a crowded room.

With less saliva to wash away bacteria, teeth and gums become slightly more vulnerable to decay and infection. Teeth also may darken slightly and become more brittle and easier to break.

With age, skin thins and becomes less elastic and more fragile and bruises more easily. Decreased production of natural oils may make skin drier and more wrinkled. Age spots can occur, and small growths called skin tags are more common.

Maintaining a healthy weight or losing weight if overweight is more difficult as one ages. Muscle mass tends to decrease with age, which leads to an increase in fat.

With age, sexual needs, patterns and performance may change. Illness or medication may affect the ability to enjoy sex. For women, vaginal dryness can make sex uncomfortable. For men, impotence may become a concern. It may take longer to get an erection, and erections may not be as firm as they used to be.

Additional disorders include as sarcopenia, diastolic dysfunction, immune deficiencies, and mobility problems. Age-related mobility problems are a very serious issue with far reaching health consequences. Age-related mobility problems lead to an increase in falls, hospitalizations, and future requirements for a caregiver, and increase the risk for depression, osteoporosis, arthritis, congestive heart failure, muscle pain, stroke, dementia and death.

While treatments exist for some symptoms of age related disorders, no treatments that address multiple functions at a molecular level are available.

### SUMMARY OF THE INVENTION

The present invention relates to therapeutic targets for aging. In particular, the present invention relates to the inhibition of the kynurenine pathway of tryptophan metabolism to extend lifespan or provide anti-aging benefits.

For example, in some embodiments, the present invention provides compositions and methods of prolonging the lifespan of a subject or providing anti-aging benefits, comprising: administering an agent that inhibits the conversion of tryptophan into kynurenine to a subject (e.g., wherein the administering prolongs the lifespan of the subject relative to the lifespan in the absence of the agent or where the one or more anti-aging benefits are achieved). In some embodiments, the agent inhibits TRY 2,3-dioxygenase 2 (TDO), indoleamine 2,3-dioxygenase (IDO) or the ATP binding cassette (ABC) transporter. For example, in some embodiments, the agent is a small molecule (e.g., alpha-methyl tryptophan or 5-methyl tryptophan), a nucleic acid (e.g., siRNA or antisense nucleic acid), an antibody, etc. In some embodiments, the administering treats, prevents reduces or retards one or more aging-associated medical or psychiatric disorders or conditions.

In some embodiments, the present invention provides a composition comprising an agent that inhibits the conversion of tryptophan into kynurenine and/or increases elimination of kynurenine or its neurotoxic metabolites (e.g., 3HK); and an agent known to be useful in treating one or more disorders associated with aging. In some embodiments, the agents are formulated in a single pharmaceutical composition.

Further embodiments of the present invention provide a method of identifying compounds that inhibit the conversion of tryptophan into kynurenine and/or increases elimination of kynurenine or its neurotoxic metabolites (e.g., 3HK) (e.g., to prolong lifespan or treat or prevent one or more aging-associated medical or psychiatric disorders), comprising: contacting a cell with a test compound; and identifying test compounds that inhibit the conversion of tryptophan into kynurenine and/or increases elimination of kynurenine or its neurotoxic metabolites (e.g., 3HK). In some embodiments, the agent inhibits TRY 2,3-dioxygenase 2 (TDO), indoleamine 2,3-dioxygenase (IDO) or the ATP binding cassette (ABC) transporter. In some embodiments, the cell is a mammalian cell (e.g., a human cell) or a drosophila cell. In some embodiments, the cell is in an animal (e.g., drosophila or a non-human mammal). In some embodiments, the agent is a small molecule, a nucleic acid (e.g., siRNA or antisense nucleic acid), an antibody, etc. In some embodiments, two or more agents working together (e.g., additively or synergistically) are evaluated. In some embodiments, aging-associated medical disorders (e.g., aging-associated medical and psychiatric disorders) include, but are not limited to, reduced cardiac output, atherosclerosis, high blood pressure, mood & cognitive changes, memory loss, vision problems, hearing loss, muscle wasting, decreased energy, abdominal fat/truncal obesity, weak bones, problems with coordination and balance, digestive problems (e.g., constipation), urinary incontinence, diabetes, thin skin and skin wrinkles, poor sleep, age related mobility problems and lessened sexual performance.

Additional embodiments are described herein.

### DESCRIPTION OF THE FIGURES

FIG. 1 shows survival time of Drosophila melanogaster (Oregon) treated with alpha-methyl (aMT) or 5-methyl (5MT) tryptophan. (p<0.0001:Logrank test).

### DEFINITIONS

To facilitate an understanding of the present invention, a number of terms and phrases are defined below:

As used herein, the term “prolonging the lifespan of a subject” refers to increasing the lifespan of a subject relative to the lifespan in the absence of the agent (e.g., relative to the projected lifespan of an individual, a population, an individual with certain diseases or lifestyle choices, etc.).

As used herein, the term “aging-associated medical and psychiatric disorders” refers to medical or psychiatric disorder typically associated with or worsened by aging. Examples include, but are not limited to, those disclosed herein.

As used herein, the term “inhibits the conversion of tryptophan into kynurenine” refers to any method of inhibiting the conversion of tryptophan into kynurenine. In some embodiments, the enzyme catalyzing tryptophan conversion into kynurenine (e.g., TRY 2,3-dioxygenase 2 (TDO) and/or indoleamine 2,3-dioxygenase (IDO)) is inhibited. In other embodiments, tryptophan tramsmembrane transport that delivers tryptophan inside the cell producing kynurenine (e.g., ATP binding cassette (ABC) transporter) is inhibited. TDO or ABC transporter may be inhibited using any suitable agent (e.g., via directly contacting TDO or ABC transporter protein, contacting TDO or ABC transporter mRNA or genomic DNA, causing conformational changes of TDO or ABC transporter polypeptides, decreasing TDO or ABC transporter protein levels, or interfering with TDO or ABC transporter interactions with signaling partners, and affecting the expression of TDO or ABC transporter target genes). Inhibitors also include molecules that indirectly regulate TDO or ABC transporter biological activity by intercepting upstream signaling molecules. In some embodiments, the inhibitor is 5-methyl tryptophan or alpha-methyl tryptophan.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., medical or psychiatric disorders of aging). Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present invention.

The term “chemical moiety” refers to any chemical compound containing at least one carbon atom. Examples of chemical moieties include, but are not limited to, aromatic chemical moieties, chemical moieties comprising sulfur, chemical moieties comprising nitrogen, hydrophilic chemical moieties, and hydrophobic chemical moieties.

The term “derivative” of a compound, as used herein, refers to a chemically modified compound wherein the chemical modification takes place either at a functional group of the compound or backbone. Such derivatives include, but are not limited to, esters of alcohol-containing compounds, esters of carboxy-containing compounds, amides of amine-containing compounds, amides of carboxy-containing compounds, imines of amino-containing compounds, acetals of aldehyde-containing compounds, ketals of carbonyl-containing compounds, and the like.

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “siRNAs” refers to small interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

### DETAILED DESCRIPTION OF THE INVENTION

The present invention relates to therapeutic targets for aging. In particular, the present invention relates to the inhibition of the kynurenine pathway of tryptophan metabolism to extend lifespan or provide anti-aging benefits.

Embodiments of the present invention provide compositions and methods of prolonging lifespan in a subject or otherwise providing anti-aging benefits by inhibiting the conversion of tryptophan (TRY) into kynurenine (KYN) and/or increases elimination of kynurenine or its neurotoxic metabolites (e.g., 3HK). For example, in some embodiments the enzyme catalyzing tryptophan conversion into kynurenine (e.g., TRY 2,3-dioxygenase 2 (TDO)) is inhibited. In other embodiments, tryptophan tramsmembrane transport that delivers tryptophan inside the cell producing kynurenine (e.g., ATP binding cassette (ABC) transporter) is inhibited.

Tryptophan (TRY) is an amino acid participating in biosynthesis of proteins and methoxyindoles (serotonin and melatonin) (Oxenkrug, 2007 Ann. N Y Acad. Sci. 1122, 35-49). TRY 2,3-dioxygenase 2 (TDO) is a rate-limiting enzyme of the major non protein route of TRY metabolism: the cleavage of indole ring of TRY with the formation of formyl-kynurenine, and, subsequently, kynurenine (KYN) (Schwarcz, 2004 Curr. Opin. Pharmacol. 4: 12-17). Since TDO is intracellular enzyme (Kudo et al., 2001 J. Physiol. 535(Pt 1), 207-215), TRY must enter cell to be available as a substrate for KYN formation. Cellular uptake of TRY is facilitated by ATP-binding cassette (ABC) transporter (MacKenzie et al., 1999 Biochim. Biophys. Acta. 1419, 173-185). Thus, besides TDO, ABC transporter is a rate-limiting factor of TRY conversion into KYN (Sullivan & Sullivan, 1980 Biochem. Genet. 18, 1109-1130). Inhibition of ABC transporter decreases entry of tryptophan into cells (and, thus, decreases substrate for kynurenine formation), and decreases entry of (already formed) kynurenine and 3-HK into cells, and facilities their elimination from the body. Therefore, it is contemplated that 5MT decreases formation of kynurenine (by preventing tryptophan entry into cell) and increases elimination of (already formed) kynurenine and 3-HK.

Animal and human studies have demonstrated that aging is associated with upregulation of TRY-KYN metabolism. Thus, plasma KYN/TRY ratio (marker of activity of KYN formation from TRY) is increased with aging (Frick et al. 2004 Clin. Biochem. 37, 684-687; Petrovaara et al. 2006 Mech. Ageing Dev. 127, 497-499; Capuron et al., 2011 Biol Psychiatry. 2011 Jan. 28). Increased formation of KYN derivative, kynurenic acid, was observed in aged rat brain (Moroni et al. 1988 Neurosci Lett. 94, 145-150; Gramsbergen et al. 1992 Brain Res. 588, 1-5) and in human serum (Urbanska et al., 2006 Pharm. Rep. 58, 507-511). The present invention is not limited to a particular mechanism. Indeed, an understanding of the mechanism is not necessary to practice the present invention. Nonetheless, it is contemplated that one of the mechanisms of aging-associated up-regulation of TRY-KYN metabolism is the increase of production of cortisol (Oxenkrug et al., 1983 Psychiatry Research, 10:125-130), an inducer of TDO.

TRY-KYN pathway and related genes were described in Drosophila melanogaster (Savvateeva-Popova et al. 2003 Adv. Exp. Med. Biol. 527, 713-722). The end product of TRY-KYN pathway in Drosophila is brown eye pigment (Tearle, 1991 Genet. Res. 57, 257-266). TDO is the rate-limiting enzyme of KYN formation from TRY in Drosophila, as in the other species. Experiments conducted during the course of development of embodiments of the present invention investigated whether prolongation of life span was associated with the slow rate of KYN formation from TRY. In difference with genetic mutations, pharmacological interventions increased not only mean survival time but maximum life span as well.

### I. Therapeutic Applications

As described above, embodiments of the present invention provide compositions and methods of inhibiting TRY conversion to KYN and/or increases elimination of kynurenine or its neurotoxic metabolites (e.g., 3HK) (e.g., by inhibiting TRY 2,3-dioxygenase 2 (TDO), indoleamine 2,3-dioxygenase (IDO) or ATP binding cassette (ABC) transporter). A number of exemplary methods of inhibiting TDO and ABC transporter are described herein. One of skill in the art recognizes that other therapeutics are suitable for use herein.

### A. Small Molecule Therapies

In other embodiments, the present invention provides small molecule inhibitors of TDO, IDO and/or ABC transporter expression or activity.

In some embodiments, the small molecule therapeutic is the TDO inhibitor, alpha-methyl tryptophan (aMT) or the ABC transporter inhibitor, 5-methyl tryptophan (5MT).

In some embodiments, the inhibitor is a mimetic, derivative, analog, stereoisomer, etc. of aMT or 5MT. Additional small molecule therapeutics can be identified using the drug screening methods described herein.

The present invention also includes pharmaceutical compositions and formulations that include the small molecule compounds of the present invention as described below.

### B. RNA Interference and Antisense Therapies

In some embodiments, the present invention targets the expression of TDO, IDO or ABC transporter. For example, in some embodiments, the present invention employs compositions comprising oligomeric antisense or RNAi compounds, particularly oligonucleotides (e.g., those described herein), for use in modulating the function of nucleic acid molecules encoding TDO, IDO or ABC transporter, ultimately modulating the amount of TDO, IDO or ABC transporter expressed.

1. RNA Interference (RNAi)

In some embodiments, RNAi is utilized to inhibit TDO, IDO or ABC transporter protein function. RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC(RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments.

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

An important factor in the design of siRNAs is the presence of accessible sites for siRNA binding. Bahoia et al., (J. Biol. Chem., 2003; 278: 15991-15997; herein incorporated by reference) describe the use of a type of DNA array called a scanning array to find accessible sites in mRNAs for designing effective siRNAs. These arrays comprise oligonucleotides ranging in size from monomers to a certain maximum, usually Corners, synthesized using a physical barrier (mask) by stepwise addition of each base in the sequence. Thus the arrays represent a full oligonucleotide complement of a region of the target gene. Hybridization of the target mRNA to these arrays provides an exhaustive accessibility profile of this region of the target mRNA. Such data are useful in the design of antisense oligonucleotides (ranging from 7mers to 25mers), where it is important to achieve a compromise between oligonucleotide length and binding affinity, to retain efficacy and target specificity (Sohail et al, Nucleic Acids Res., 2001; 29(10): 2041-2045). Additional methods and concerns for selecting siRNAs are described for example, in WO 05054270, WO05038054A1, WO03070966A2, J Mol. Biol. 2005 May 13; 348(4):883-93, J Mol. Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection of siRNAs.

In some embodiments, the present invention utilizes siRNA including blunt ends (See e.g., US20080200420, herein incorporated by reference in its entirety), overhangs (See e.g., US20080269147A1, herein incorporated by reference in its entirety), locked nucleic acids (See e.g., WO2008/006369, WO2008/043753, and WO2008/051306, each of which is herein incorporated by reference in its entirety). In some embodiments, siRNAs are delivered via gene expression or using bacteria (See e.g., Xiang et al., Nature 24: 6 (2006) and WO06066048, each of which is herein incorporated by reference in its entirety).

In other embodiments, shRNA techniques (See e.g., 20080025958, herein incorporated by reference in its entirety) are utilized. A small hairpin RNA or short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. shRNA is transcribed by RNA polymerase III.

The present invention also includes pharmaceutical compositions and formulations that include the RNAi compounds of the present invention as described below.

2. Antisense

In other embodiments, TDO, IDO or ABC transporter protein expression is modulated using antisense compounds that specifically hybridize with one or more nucleic acids encoding TDO, IDO or ABC transporter. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of TDO, IDO or ABC transporter. In the context of the present invention, “modulation” means either an increase (stimulation) or a decrease (inhibition) in the expression of a gene. For example, expression may be inhibited to prevent symptoms related to disorders of aging and thus prolong lifespan.

The present invention also includes pharmaceutical compositions and formulations that include the antisense compounds of the present invention as described below.

### C. Genetic Therapy

The present invention contemplates the use of any genetic manipulation for use in modulating the expression of TDO, IDO or ABC transporter. Examples of genetic manipulation include, but are not limited to, gene knockout (e.g., removing the TDO, IDO or ABC transporter gene from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct). Genetic therapy may also be used to deliver siRNA or other interfering molecules that are expressed in vivo (e.g., upon stimulation by an inducible promoter (e.g., an androgen-responsive promoter)).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of tissues in animal models. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subjects in a variety of ways. For example, in some embodiments of the present invention, vectors are administered using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

### D. Pharmaceutical Compositions

The compounds are preferably employed for therapeutic uses in combination with a suitable pharmaceutical carrier. Such compositions comprise an effective amount of the compound, and a pharmaceutically acceptable carrier or excipient. The formulation is made to suit the mode of administration. Pharmaceutically acceptable carriers are determined in part by the particular composition being administered, as well as by the particular method used to administer the composition. Accordingly, there is a wide variety of suitable formulations of pharmaceutical compositions containing the nucleic acids some of which are described herein.

The compounds may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation. The compound may also be encapsulated in suitable biocompatible microcapsules, microparticles or micro spheres formed of biodegradable or non-biodegradable polymers or proteins or liposomes for targeting to cells. Such systems are well known to those skilled in the.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, with an added preservative.

Preparations include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation can be found in Remington's Pharmaceutical Sciences, Mack Publishing Co., Easton, Pa. Those of skill in the art can readily determine the various parameters for preparing and formulating the compositions without resort to undue experimentation.

The compound alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like. For administration by inhalation, the compounds are conveniently delivered in the form of an aerosol spray presentation from pressurized packs or a nebulizer, with the use of a suitable propellant.

In some embodiments, the compound described above may include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. In one embodiment, the compounds are conjugated to lipophilic groups like cholesterol and laurie and lithocholic acid derivatives with C32 functionality to improve cellular uptake. For example, cholesterol has been demonstrated to enhance uptake and serum stability of siRNA in vitro Lorenz, et al., Bioorg. Med. Chem. Lett. 14(19):4975-4977 (2004)) and in vivo (Soutschek, et al., Nature 432(7014):173-178 (2004)). In addition, it has been shown that binding of steroid conjugated oligonucleotides to different lipoproteins in the bloodstream, such as LDL, protect integrity and facilitate biodistribution (Rump, et al., Biochem. Pharmacol. 59 (11):1407-1416 (2000)). Other groups that can be attached or conjugated to the compound described above to increase cellular uptake, include acridine derivatives; cross-linkers such as psoralen derivatives, azidophenacyl, proflavin, and azidoproflavin; artificial endonucleases; metal complexes such as EDTA-Fe(II) and porphyrin-Fe(II); alkylating moieties; nucleases such as alkaline phosphatase; terminal transferases; abzymes; cholesteryl moieties; lipophilic carriers; peptide conjugates; long chain alcohols; phosphate esters; radioactive markers; non-radioactive markers; carbohydrates; and polylysine or other polyamines.

U.S. Pat. No. 6,919,208 to Levy, et al., herein incorporated by reference, also described methods for enhanced delivery. These pharmaceutical formulations may be manufactured in a manner that is itself known, e.g., by means of conventional mixing, dissolving, granulating, levigating, emulsifying, encapsulating, entrapping or lyophilizing processes.

Compositions can be administered by a number of routes including, but not limited to: oral, intravenous, intraperitoneal, intramuscular, transdermal, subcutaneous, topical, sublingual, or rectal means. Compounds can also be administered via liposomes. Such administration routes and appropriate formulations are generally known to those of skill in the art.

The particular mode selected will depend of course, upon factors such as the particular formulation, the severity of the state of the subject being treated, and the dosage required for therapeutic efficacy. As generally used herein, an “effective amount” is that amount which is able to treat one or more symptoms of aging related disorders, reverse the progression of one or more symptoms of aging related disorders, halt the progression of one or more symptoms of aging related disorders, or prevent the occurrence of one or more symptoms of aging related disorders in a subject to whom the formulation is administered, as compared to a matched subject not receiving the compound.

The actual effective amounts of compound can vary according to the specific compound or combination thereof being utilized, the particular composition formulated, the mode of administration, and the age, weight, condition of the individual, and severity of the symptoms or condition being treated.

Any acceptable method known to one of ordinary skill in the art may be used to administer a formulation to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. The composition can be injected intraderinally for treatment or prevention of aging related disorders, for example. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

Other delivery systems suitable include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these.

Use of a long-term release implant may be particularly suitable in some embodiments. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

Dosages for a particular individual can be determined by one of ordinary skill in the art using conventional considerations, (e.g. by means of an appropriate, conventional pharmacological protocol). A physician may, for example, prescribe a relatively low dose at first, subsequently increasing the dose until an appropriate response is obtained. The dose administered to a individual is sufficient to effect a beneficial therapeutic response in the individual over time, or, e.g., to reduce symptoms, or other appropriate activity, depending on the application. The dose is determined by the efficacy of the particular formulation, and the activity, stability or serum half-life of the therapeutic employed and the condition of the individual, as well as the body weight or surface area of the individual to be treated. The size of the dose is also determined by the existence, nature, and extent of any adverse side-effects that accompany the administration of a particular vector, formulation, or the like in a particular individual.

### E. Co-Administration

In some embodiments, the present invention provides combination therapies. The formulations described herein can supplement treatment conditions by any known conventional therapy, including, but not limited to, antibody administration, vaccine administration, administration of cytotoxic agents, natural amino acid polypeptides, nucleic acids, nucleotide analogues, and biologic response modifiers. Two or more combined compounds may be used together or sequentially. For example, agents can also be administered in therapeutically effective amounts as a portion of an anti-age-related disorder cocktail.

For example, in some embodiments, one or more of the therapeutic compositions described herein are combined with a known anti-aging agent. In some embodiments, compounds for co-administration are formulated together in a composition (e.g., pill, tablet, liquid, injectible formulation, etc). In other embodiments, compounds are separately formulated but administered to the same subject.

### II. Antibodies

The present invention provides isolated antibodies. In some embodiments, the present invention provides monoclonal antibodies that specifically bind to an isolated polypeptide comprised of at least five amino acid residues of TDO, IDO or ABC transporter. These antibodies find use in the therapeutic and drug screening methods described herein.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

### III. Drug Screening Applications

In some embodiments, the present invention provides drug screening assays (e.g., to screen for drugs that inhibit TDO, IDO or ABC transporter). In some embodiments, the screening methods of the present invention utilize TDO, IDO or ABC transporter or a measure of their activity or expression. For example, in some embodiments, the present invention provides methods of screening for compounds that alter (e.g., decrease) the expression or activity of TDO, IDO or ABC transporter. The compounds or agents may interfere with transcription, by interacting, for example, with the promoter region. The compounds or agents may interfere with mRNA produced from TDO, IDO or ABC transporter (e.g., by RNA interference, antisense technologies, etc.). The compounds or agents may interfere with pathways that are upstream or downstream of the biological activity of TDO, IDO or ABC transporter. In some embodiments, candidate compounds are antisense or interfering RNA agents (e.g., oligonucleotides) directed against TDO or ABC transporter. In other embodiments, candidate compounds are antibodies or small molecules that specifically bind to an TDO, IDO or ABC transporter regulator or expression products of the present invention and inhibit its biological function.

In one screening method, candidate compounds are evaluated for their ability to alter TDO, IDO or ABC transporter expression by contacting a compound with a cell expressing TDO, IDO or ABC transporter and then assaying for the effect of the candidate compounds on expression. In some embodiments, the effect of candidate compounds on expression of an TDO, IDO or ABC transporter gene is assayed for by detecting the level of TDO, IDO or ABC transporter mRNA expressed by the cell. mRNA expression can be detected by any suitable method.

In other embodiments, the effect of candidate compounds on expression of TDO, IDO or ABC transporter genes is assayed by measuring the level of polypeptide encoded by TDO, IDO or ABC transporter. The level of polypeptide expressed can be measured using any suitable method, including but not limited to, those disclosed herein.

Specifically, the present invention provides screening methods for identifying modulators, i.e., candidate or test compounds or agents (e.g., proteins, peptides, peptidomimetics, peptoids, small molecules or other drugs) which bind to TDO, IDO or ABC transporter, have an inhibitory (or stimulatory) effect on, for example, TDO, IDO or ABC transporter expression or TDO, IDO or ABC transporter activity, or have a stimulatory or inhibitory effect on, for example, the expression or activity of a TDO, IDO or ABC transporter substrate. Compounds thus identified can be used to modulate the activity of target gene products (e.g., TDO, IDO or ABC transporter) either directly or indirectly in a therapeutic protocol, to elaborate the biological function of the target gene product, or to identify compounds that disrupt normal target gene interactions. Compounds that inhibit the activity or expression of TDO, IDO or ABC transporter are useful in the treatment of aging related disorders.

In one embodiment, the invention provides assays for screening candidate or test compounds that are substrates of an TDO, IDO or ABC transporter protein or polypeptide or a biologically active portion thereof. In another embodiment, the invention provides assays for screening candidate or test compounds that bind to or modulate the activity of an TDO or ABC transporter protein or polypeptide or a biologically active portion thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

### EXPERIMENTAL

The following examples are provided in order to demonstrate and further illustrate certain preferred embodiments and aspects of the present invention and are not to be construed as limiting the scope thereof.

### Example 1

### Methods

Wild type Oregon-R flies were maintained at 23° C. on a standard Drosophila medium consisting of sugar, yeast, agar and semolina. Two concentrations of aMT (alpha-dl-methyl tryptophan) (0.46 mM or 18.3 mM) or 5MT (5-methyl-dl-tryptophan) (2.4 mM and 18.mM) (Sigma Aldrich Chemical Co, USA) were added to nutrition medium of experimental groups. To examine lifespan, 1-day old adult flies were collected and then regularly transferred to fresh medium every 3-4 days. The number of dead flies was recorded at the time of transfer.

### Statistics

The data were analyzed using two ways ANOVA and Log rank test.

### Results

### Effect of Alpha-Methyl Tryptophan

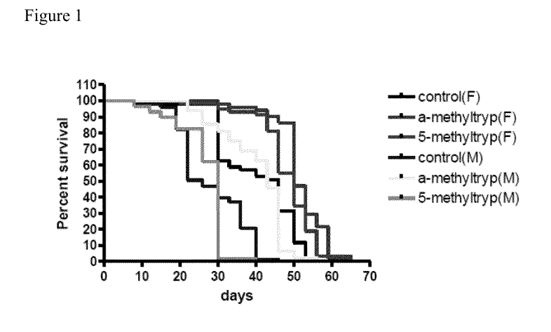

Treatment with aMT (0.46 mM) did not affect the life span of Drosophila. Treatment with higher concentration of aMT (18.3 mM) increased mean survival time by 27% for females and by 42% for males (p<0.0001, two way ANOVA) (Table 1). Treatment with high concentration of aMT (18.3 mM) increased maximum life span in both female and male flies by 23% and 21% (resp.)(p<0.0001, two way ANOVA) (Table 1). Maximum life span of female control group was 53 days. 25% (13 out of 51) female flies treated with high concentrations of aMT survived longer than 53 days (up to 65 days) (FIG. 1). Maximum life span of 72 out of 73 male control flies was 40 days. Only one control fly (1.3%) lived up to 46 days while 60% (29 out of 40) male flies treated with high concentrations of aMT survived longer than 40 days (up to 56 days) (FIG. 1).

### Effect of 5-Methyl Tryptophan.

Treatment with 5MT (2.3 mM) did not affect the life span of Drosophila. Treatment with high concentration of aMT (18.3 mM) did not affect life span of male flies (Table 1). Treatment with higher concentration of 5MT (34.5 mM) increased mean survival time (by 21%) and maximum life span (by 23%) of female flies (p<0.0001, two way ANOVA) (Table 1). Maximum life span of female control group was 53 days. 19% (11 out of 58) female flies treated with high concentrations of 5MT survived longer than 53 days (up to 65 days).

TABLE 1
Inhibitors of tryptophan - kynurenine metabolism and life span
of Drosophila melanogaster (Oregon).

| | FEMALE | | | MALE | | |
|---|---|---|---|---|---|---|
| | Life span (days) | | | Life span (days) | | |
| | Control | aMT | 5MT | Control | aMT | 5MT |
| | (N = 50) | (N = 51) | (N = 58) | (N = 73) | (N = 48) | (N = 58) |
| Mean | 40.1 | 51.7* | 48.5* | 28.3 | 40.2* | 26.7 |
| Std Err | 1.4 | 0.9 | 1.0 | 1.0 | 1.2 | 0.8 |
| Median | 44.5 | 50 | 50 | 26 | 43* | 30 |
| Maximum | 53 | 65* | 65* | 46** | 56* | 40 |

Concentrations of aMT (18.3 mM) and 5MT (18.3 mM);
*)increase in days in comparison with control group; p < 0.0001, two-way ANOVA
**)Maximum life span of 72 out of 73 male control flies was 40 days. Only one control fly (1.3%) lived up to 46 days

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

We claim:

1. A method of prolonging the lifespan of a subject or preventing or reducing age-related symptoms, comprising:
administering an agent that inhibits the conversion of tryptophan into kynurenine and/or increases elimination of kynurenine to a subject,
wherein said administering prolongs the lifespan of said subject relative to the lifespan in the absence of said agent or wherein age-related symptoms are reduced or prevented.

2. The method of claim 1, wherein said agent inhibits TRY 2,3-dioxygenase 2 (TDO) or indoleamine 2,3-dioxygenase (IDO).

3. The method of claim 2, wherein said agent is alpha-methyl tryptophan.

4. The method of claim 1, wherein said agent inhibits the ATP binding cassette (ABC) transporter.

5. The method of claim 4, wherein said agent is 5-methyl tryptophan.

6. The method of claim 1, wherein said administering treats or prevents one or more aging-associated medical or psychiatric disorders.

7. The method of claim 1, wherein said agent is selected from the group consisting of a small molecule, a nucleic acid, and an antibody.

8. A method of identifying compounds that inhibit the conversion of tryptophan into kynurenine and/or increases elimination of kynurenine, comprising:
a) contacting a cell with a test compound; and
b) identifying test compounds that inhibit the conversion of tryptophan into kynurenineor increase elimination of kynurenine.

9. The method of claim 8, wherein said agent inhibits TRY 2,3-dioxygenase 2 (TDO) or indoleamine 2,3-dioxygenase (IDO).

10. The method of claim 8, wherein said agent inhibits the ATP binding cassette (ABC) transporter.

11. The method of claim 8, wherein said cell is a mammalian cell.

12. The method of claim 11, wherein said cell is a human cell.

13. The method of claim 8, wherein said cell is a drosophila cell.

14. The method of claim 8, wherein said cell is in an animal.

15. The method of claim 14, wherein said animal is drosophila.

16. The method of claim 14, wherein said animal is a non-human mammal.

17. The method of claim 8, wherein said agent is selected from the group consisting of a small molecule, a nucleic acid, and an antibody.

18. A composition, comprising:
a) a first agent that inhibits the conversion of tryptophan into kynurenine and/or increases elimination of kynurenine; and
b) a second agent known to be useful in the treatment of one or more disorders of aging.

19. The composition of claim 18, wherein said first agent inhibits TRY 2,3-dioxygenase 2 (TDO) or indoleamine 2,3-dioxygenase (IDO).

20. The composition of claim 19, wherein said first agent is alpha-methyl tryptophan.

21. The composition of claim 18, wherein said first agent inhibits the ATP binding cassette (ABC) transporter.

22. The composition of claim 21, wherein said agent is 5-methyl tryptophan.
